(19) United States
(12) Patent Application Publication
(10) Pub. No.: US 2017/0042513 A1
(43) Pub. Date: Feb. 16, 2017

(54) ULTRASONIC DIAGNOSING DEVICE

(71) Applicant: HITACHI, LTD., Tokyo (JP)

(72) Inventors: Kagami MIYAJI, Kanagawa (JP); Keiichi ITATANI, Kanagawa (JP); Hajime SAKASHITA, Tokyo (JP); Tomohide NISHIYAMA, Tokyo (JP)

(73) Assignee: Hitachi, Ltd., Tokyo (JP)

(21) Appl. No.: 15/307,025

(22) PCT Filed: Apr. 9, 2015
(86) PCT No.: PCT/JP2015/061097

§ 371 Date: Oct. 27, 2016

(30) Foreign Application Priority Data
Apr. 28, 2014 (JP) 2014-092525

(51) Int. Cl. A61B 8/08; A61B 8/00; A61B 8/06

(52) U.S. Cl. CPC A61B 8/5223; A61B 8/488; A61B 8/06; A61B 8/5207; A61B 8/0883; A61B 8/461; A61B 8/467

(57) ABSTRACT

An image forming processing unit forms image data for the interior of a body on the basis of a received ultrasonic signal. A Doppler processing unit generates Doppler information for the body interior on the basis of the receiving signals of ultrasonic waves. A velocity vector calculating unit generates velocity information (velocity vector) for blood flow on the basis of the Doppler information for the body interior. A region of interest setting unit sets a region of interest corresponding to a heart cavity in the image data. An energy calculating unit calculates the amount of energy lost in the blood flow in the heart cavity on the basis of the blood flow velocity information in the region of interest corresponding to the heart cavity.

### TECHNICAL FIELD

The present invention relates to an ultrasonic diagnostic device, and more particularly to a technique of obtaining diagnosis information concerning a heart.

### BACKGROUND

Techniques known to the art include obtaining, based on a received signal generated by transmitting and receiving ultrasonic waves with respect to a fluid such as blood flow, diagnosis information regarding the fluid. Patent Document 1, for example, describes a technique of obtaining, based on a received signal (echo data) obtained by transmitting and receiving ultrasonic waves with respect to fluid within an organism, two-dimensional velocity vectors regarding the fluid at a plurality of points within an observation plane. It is possible to extract, from a distribution of the two-dimensional velocity vectors at a plurality of points within the observation plane, diagnosis information such as a streamline representing a flow of fluid. Application of such information to diagnosis of a heart, for example, is expected.

### CITATION LIST

### Patent Literature

Patent Document 1: JP-2013-192643 A

### SUMMARY

### Technical Problem

In consideration of the background art described above, the inventors of the present application have repeated research and development concerning techniques of obtaining diagnosis information of a heart by utilizing ultrasonic waves.

The present invention was achieved in the process of the research and development, and is directed to providing an improved technique for obtaining diagnosis information concerning a heart by utilizing ultrasonic waves.

### Solution to Problem

To achieve the above object, an ultrasonic diagnostic device in accordance with a preferred aspect includes a probe configured to transmit and receive an ultrasonic wave, a transmitter and receiver unit configured to control the probe to obtain a received signal of an ultrasonic wave from an interior of an organism, a Doppler processing unit configured to obtain Doppler information of the interior of the organism based on the received signal of an ultrasonic wave, a velocity information generating unit configured to obtain velocity information of blood flow based on the Doppler information of the interior of the organism, an image forming processing unit configured to obtain image data concerning the interior of the organism based on the received signal of an ultrasonic wave, a region of interest setting unit configured to set, in the image data, a region of interest corresponding to a heart cavity, an energy computation unit configured to calculate an amount of energy lost (an amount of energy loss) within blood flow in the heart cavity based on the velocity information of blood flow within the region of interest corresponding to the heart cavity, and a display processing unit configured to form a display image showing the amount of lost energy which is calculated.

In the above configuration, the velocity information of blood flow refers to information regarding a partial or whole motion of blood flow, and specifically includes, for example, a velocity vector indicating the velocity and direction of each portion within blood flow or a shift vector indicating an amount and direction of shift of each portion. While the velocity vector and shift vector of blood flow are preferably derived by utilizing the technique described, for example, in Patent Document 1 (two-dimensional velocity vector distribution), the velocity vector and the like may also be obtained by utilizing other known techniques.

The amount of energy which is lost within the blood flow refers, for example, to an amount of kinetic energy which has been converted to thermal energy due to friction and the like within blood flow and lost. This amount of lost energy can be expected to serve as an evaluation value for evaluating the function of the heart and the blood vessel. For example, the kinetic energy of blood flow within a heart cavity lowers by an amount corresponding to the amount of energy lost in blood flow of the heart cavity. It can therefore be assumed that a large quantity of kinetic energy would be applied from the heart to the blood flow to maintain the flow rate of the blood flow, imposing a significant burden on the heart (cardiac burden). It can also be assumed that the blood flow flowing into or out of the heart cavity having small kinetic energy cannot maintain a sufficient flow rate.

The above device can calculate the amount of energy lost within blood flow in the heart cavity and use the calculated amount of the energy loss as an evaluation value for evaluating the function of the heart and the blood vessel. This enables evaluation of the function of the heart and the blood vessel based on the amount of energy loss, and more preferably enables comprehensive diagnosis of the function of the heart and the blood vessel based on a plurality of evaluation values by using, as additional evaluation values, kinetic energy of blood flow flowing into the heart cavity and kinetic energy of blood flow flowing out of the heart cavity, for example.

In some preferable specific examples, the energy computation unit may be configured to calculate the amount of lost energy for each of a plurality of sample points within the region of interest corresponding to the heart cavity.

In some preferable specific examples, the display processing unit may be configured to apply, to the image data concerning the interior of the organism at each of the sample points within the region of interest, display processing in accordance with the amount of lost energy of the sample point, to thereby form a display image showing a state of distribution of the amount of lost energy within the heart cavity.

In some preferable specific examples, the energy computation unit may be configured to calculate a sum total of the amount of lost energy within the region of interest, based on the amount of lost energy at the plurality of sample points within the region of interest corresponding to the heart cavity, and the display processing unit may be configured to form a graph indicating, over a plurality of time phases, the amount of lost energy which is calculated for each time phase.

In some preferable specific examples, the region of interest setting unit may be configured to designate a region, in the image data, enclosed by a cavity line which is set as an outer edge of the heart cavity, as the region of interest.

In some preferable specific examples, the region of interest setting unit may be configured to designate a region, in the image data, enclosed by an inflow line which is set in a flow path of blood flow flowing in the heart cavity, an outflow line which is set in a flow path of blood flow flowing out of the heart cavity, and a cavity line which is set as an outer edge of the heart cavity, as the region of interest.

In some preferable specific examples, the energy computation unit may be configured to calculate inflow energy that is kinetic energy of blood flow flowing into the heart cavity, based on the velocity information of blood flow corresponding to an inflow line which is set in a flow path of blood flow flowing into the heart cavity and calculate outflow energy that is kinetic energy of blood flow flowing out of the heart cavity, based on the velocity information of blood flow corresponding to an outflow line which is set in a flow path of blood flow flowing out of the heart cavity, and the display processing unit may be configured to form a graph showing at least one of the inflow energy and the outflow energy (the inflow energy and/or the outflow energy) calculated for each time phase, over a plurality of time phases.

In some preferable specific examples, the velocity information generating unit may be configured to generate velocity information of at least one of blood flow and cardiac muscle (blood flow and/or cardiac muscle) on a cavity line which is set as an outer edge of the heart cavity, based on at least one of the Doppler information and the image data (the Doppler information and/or the image data) concerning the interior of the organism, and the energy computation unit may be configured to calculate kinetic energy to be exerted on the heart cavity, based on the velocity information on the cavity line.

In some preferable specific examples, the velocity information generating unit may be configured to generate the velocity information of blood flow or cardiac muscle for each of a plurality of sample points on the cavity line, and the energy computation unit may be configured to calculate a sum total of the kinetic energy to be exerted on the heart cavity based on the velocity information at the plurality of sample points on the cavity line.

In some preferable specific examples, the energy computation unit is configured to calculate, from the velocity information at the plurality of sample points on the cavity line, a sum total of the kinetic energy based on the velocity information toward the inside of the heart cavity and a sum total of the kinetic energy based on the velocity information toward the outside of the heart cavity.

To achieve the above object, an information processing device in accordance with a preferred aspect includes a velocity information generating unit configured to obtain velocity information of blood flow based on Doppler information of an interior of an organism obtained using an ultrasonic wave, a region of interest setting unit configured to set a region of interest corresponding to a heart cavity in image data concerning the organism obtained using an ultrasonic wave, and an energy computation unit configured to calculate an amount of energy lost within blood flow in the heart cavity based on the velocity information of blood flow within the region of interest corresponding to the heart cavity.

The information processor described above can be implemented, for example, by a computer. For example, it is possible to cause a computer to function as the information processor described above using a program which causes the computer to implement a velocity information generation function to obtain velocity information of blood flow based on Doppler information within an organism obtained using ultrasonic waves, a region of interest setting function to set a region of interest corresponding to a heart cavity within image data regarding the organism obtained using ultrasonic waves, and an energy computation function to calculate the amount of energy lost within the blood flow of the heart cavity based on the velocity information of the blood flow within the region of interest corresponding to the heart cavity. The program may be stored in a computer-readable storage medium such as a disk or a memory and provided to the computer via the storage medium or may be provided to the computer via an electronic communication line such as the Internet.

### Advantageous Effects of Invention

The present invention is directed to providing an improved technique for obtaining diagnosis information of a heart using ultrasonic waves. In accordance with a preferable aspect of the invention, the amount of energy lost within blood flow of the heart cavity is calculated and can be used as an evaluation value for evaluating the function of the heart and the blood vessel. This enables evaluation of the function of the heart and the blood vessel based on the amount of energy loss, and more preferably enables comprehensive diagnosis of the function of the heart and the blood vessel based on a plurality of evaluation values including, as additional evaluation values, kinetic energy of blood flow flowing in the heart cavity and kinetic energy of blood flow flowing out of the heart cavity, for example.

### BRIEF DESCRIPTION OF DRAWINGS

FIG. 1 is a diagram illustrating a whole structure of an ultrasonic diagnostic device according to an embodiment of the present invention.

FIG. 2 is a diagram illustrating a specific example region of interest corresponding to a heart cavity.

FIG. 3 is a diagram for explaining a calculation example of inflow energy and outflow energy.

FIG. 4 is a specific example graph showing a change in the energy loss with time, for example.

FIG. 5 is a specific example display image regarding a distribution of the energy loss.

FIG. 6 is a diagram illustrating a specific example for calculating kinetic energy from a cavity line.

FIG. 7 is a diagram illustrating a specific example graph showing a change in the total kinetic energy with time, for example.

### DESCRIPTION OF EMBODIMENTS

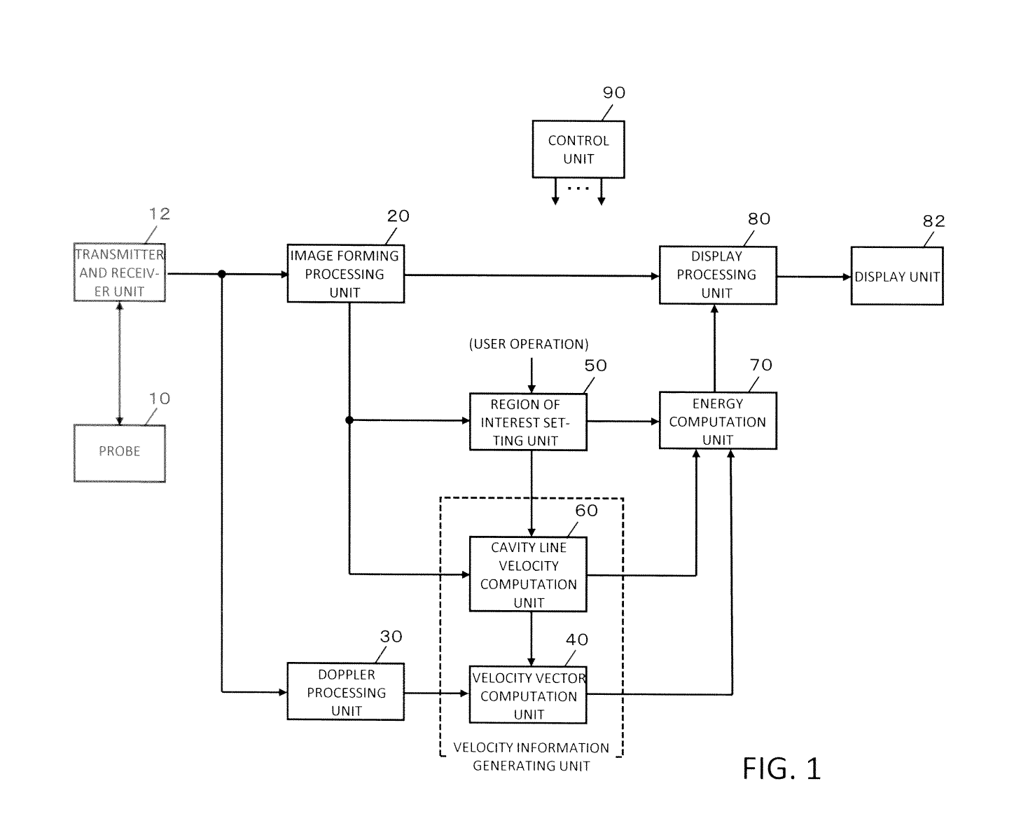

FIG. 1 is a diagram illustrating a whole structure of an ultrasonic diagnostic device according to a preferable embodiment of the present invention. The ultrasonic diagnostic device illustrated in FIG. 1 has a function to generate diagnosis information regarding a heart, and can particularly calculate an amount of energy lost within blood flow in a heart cavity, for example.

A probe 10 is an ultrasound probe which transmits and receives ultrasonic waves to and from a region within an organism including the heart. The probe 10 includes a plurality of transducer elements, which are electronically scan-controlled to scan an ultrasound beam within a space including the heart. The probe 10 is held by a user (examiner) such as a doctor and is used in contact with a body surface of an examinee. The probe 10 may be inserted into a body cavity of the examinee for use or may be a probe which performs both electronic scanning and mechanical scanning.

A transmitter and receiver unit 12 has a function as a transmitting beam former and a received beam former. Specifically, the transmitter and receiver unit 12 outputs a transmitting signal to each of the plurality of transducer elements of the probe 10 to thereby form a transmitting beam, and further applies phase alignment and summation processing, for example, to a plurality of received signals obtained from the plurality of transducer elements to thereby form a received beam. Thus, an ultrasound beam (the transmitting beam and the received beam) is scanned within a scanning plane and a received signal is formed along the ultrasound beam. To obtain the received signal of ultrasonic waves, an ultrasound beam may be scanned within a three-dimensional space in a stereoscopic manner or a technique, such as transmission aperture synthesis, may be used.

An image forming processing unit 20, based on the received signal of ultrasonic waves obtained from the scanning plane, forms data for an ultrasound image (image data). The image forming processing unit 20 applies detection processing, filter processing, AD conversion processing, and other processing to the received signal of ultrasonic waves, for example, to form frame data for a B-mode image. Of course, image data concerning known ultrasound image other than a B-mode image may be formed.

A Doppler processing unit 30 measures a Doppler shift amount contained in the received signal obtained along the ultrasound beam. The Doppler processing unit 30 specifically measures a Doppler shift occurring within the received signal of ultrasonic waves due to blood flow by using known Doppler processing, for example, to obtain velocity information concerning the blood flow in the ultrasound beam direction.

A velocity vector computation unit 40, based on the velocity information concerning the blood flow in the ultrasound beam direction, forms a distribution of two-dimensional velocity vectors within the scanning plane. The velocity vector computation unit 40 uses, in addition to the velocity information concerning the blood flow in the ultrasound beam direction, motion information of the cardiac wall, to obtain two-dimensional velocity vectors of the blood flow at each location within the scanning plane, as described in Patent Document 1 (JP 2013-192643 A), for example.

The two-dimensional velocity vectors distribution within the scanning plane can be formed from one-dimensional velocity information along the ultrasound beam using various known methods. Of course, the two-dimensional velocity vectors may be formed by forming two ultrasound beams in different directions and obtaining velocity information from each of the two ultrasound beams.

The velocity vector computation unit 40 obtains a velocity vector for each of a plurality of sample points in a calculation coordinate system corresponding to a space in which the ultrasonic waves are transmitted and received. For example, the calculation coordinate system is represented by an xyz orthogonal coordinate system, and a velocity vector is obtained for each sample point within an xy plane corresponding to the scanning plane of ultrasonic waves to form the distribution of two-dimensional velocity vectors. The distribution of two-dimensional velocity vectors may be formed in a scanning coordinate system corresponding to scanning of ultrasonic waves, such as an rθ coordinate system with the depth direction r of beams and the scanning direction θ of beams.

A region of interest setting unit 50 sets a region of interest corresponding to a heart cavity in the image data obtained by the processing performed by the image forming processing unit 20. The region of interest setting unit 50 designates a region within the image data enclosed by a cavity line which is set as the outer edge of the heart cavity, an inflow line which is set in a flow path of blood flow flowing into the heart cavity, and an outflow line which is set in a flow path of blood flow flowing out of the heart cavity, as the region of interest. The processing in the region of interest setting unit 50 and specific example region of interest will be described in detail below.

A cavity line velocity computation unit 60, based on the image data formed by the image forming processing unit 20, generates velocity information of cardiac muscle (cardiac wall) on the cavity line. The cavity line velocity computation unit 60 generates the velocity information of the cardiac muscle for each of a plurality of sample points on the cavity line.

The cavity line velocity computation unit 60, for example, performs pattern matching using a correlation operation based on pixel values (luminance values, for example) of the image data, for example, between frames of the image data obtained over a plurality of frames, and tracks, for each sample point on the cavity line, a location where the sample point is shifted within a two-dimensional plane, concerning the plurality of frames. Thus, two-dimensional shift information is obtained for each sample point, so that the two-dimensional velocity vectors can be calculated based on a shift amount between frames and the time between frames. When the image data corresponds to the xy orthogonal coordinate system, velocity vectors in the xy orthogonal coordinate system would be calculated, and when the image data corresponds to the rθ coordinate system, velocity vectors in the rθ coordinate system would be calculated.

When the velocity vector computation unit 40 uses the technique described in Patent Document 1 to obtain the two-dimensional velocity vectors of blood flow, motion information of the cardiac wall would be used. In this case, the velocity vector concerning each sample point on the cavity line calculated in the cavity line velocity computation unit 60 may be used as the motion information.

As described above, the velocity vector computation unit 40 and the cavity line velocity computation unit 60 function as a velocity information generating unit to generate the velocity information (velocity vectors) of blood flow within the heart cavity and the velocity information (velocity vectors) of cardiac muscle (cardiac wall).

An energy computation unit 70, based on the velocity information of blood flow within a region of interest corresponding to the heart cavity, calculates an amount of energy lost in the blood flow of the heart cavity. The energy computation unit 70 further calculates inflow energy which is kinetic energy of blood flow flowing into the heart cavity, based on the velocity information of blood flow corresponding to the inflow line which is set in the flow path of the blood flow flowing into the heart cavity, and calculates outflow energy which is kinetic energy of blood flow flowing out of the heart cavity, based on the velocity information of blood flow corresponding to the outflow line which is set in the flow path of the blood flow flowing out of the heart cavity. The energy computation unit 70 further calculates kinetic energy to be exerted on the heart cavity, based on the velocity information on the cavity line. Specific example processing performed by the energy computation unit 70 will be described in detail below.

A display processing unit 80 forms a display image, based on the image data of an ultrasound image obtained by the image forming processing unit 20 and the calculated values concerning the energy obtained by the energy computation unit 70, for example. The display image formed in the display processing unit 80 is displayed on a display unit 82.

A control unit 90 controls the whole ultrasonic diagnostic device illustrated in FIG. 1. The ultrasonic diagnostic device of FIG. 1 may preferably include an operation device, such as a mouse, a keyboard, a trackball, a touch panel, or a joy stick. An instruction received by a user via the operation device is also reflected in the whole control performed by the control unit 90.

Among the elements (each unit designated by a reference numeral) illustrated in FIG. 1, the transmitter and receiver unit 12, the image forming processing unit 20, the Doppler processing unit 30, the velocity vector computation unit 40, the region of interest setting unit 50, the cavity line velocity computation unit 60, the energy computation unit 70, and the display processing unit 80 may be implemented by using hardware such as an electrical or electronic circuit or a processor, for example, and a device such as a memory may be used for the implementation. The function corresponding to each unit described above may be implemented by cooperation of hardware such as a CPU, a processor, or a memory, and software (program) which regulates the operation of the CPU or the processor. A specific example of the display unit 82 may include a liquid crystal display, for example. The control unit 90 can be implemented by cooperation of hardware such as a CPU, a processor, or a memory, and software (program) which regulates the operation of the CPU or the processor.

The ultrasonic diagnostic device illustrated in FIG. 1 has been summarized as above. Specific example functions to be implemented by the ultrasonic diagnostic device of FIG. 1 will now be described in detail. In the following description concerning the elements (sections denoted by reference numerals) illustrated in FIG. 1, the reference numerals in FIG. 1 will be used.

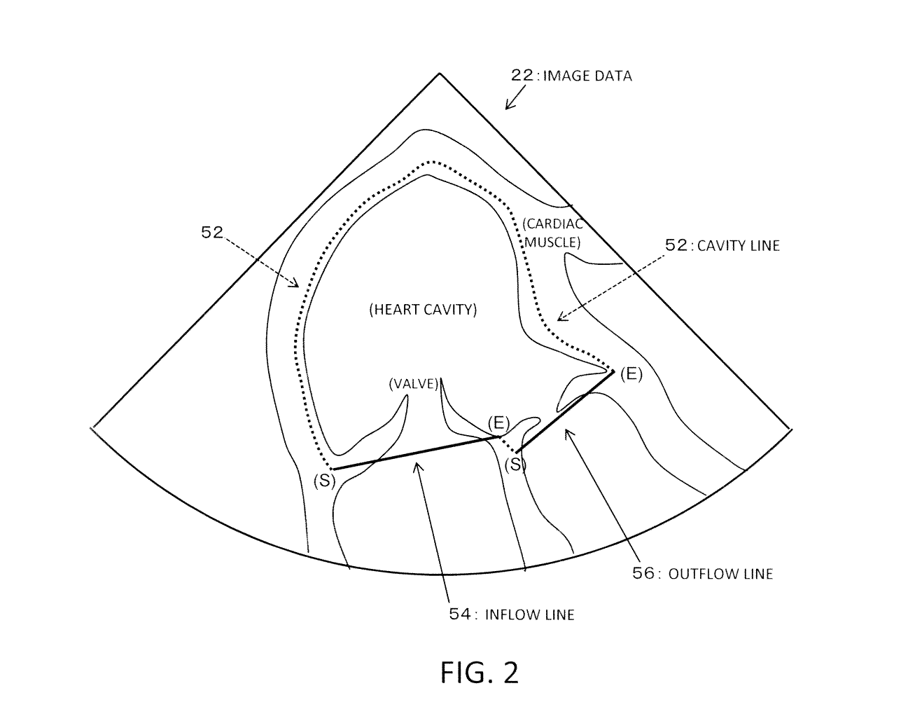

FIG. 2 is a diagram illustrating a specific example region of interest corresponding to the heart cavity. FIG. 2 illustrates specific example image data 22 obtained by the image forming processing unit 20. The image data 22 includes a heart cavity enclosed by cardiac muscle and valves.

The region of interest setting unit 50 designates a region within the image data 22 enclosed by a cavity line 52 which is set as the outer edge of the heart cavity, an inflow line 54 which is set in a flow path of blood flow flowing into the heart cavity, and an outflow line 56 which is set in a flow path of blood flow flowing out of the heart cavity, as a region of interest.

The cavity line 52 is formed based on a plurality of trace points corresponding to the outer edge of the heart cavity. For example, a user, such as a doctor, sets, using an operation device, a plurality of trace points on or near the boundary between the heart cavity and the cardiac muscle, on a display image corresponding to the image data 22 indicated on the display unit 82. The plurality of trace points set by the user are then connected by interpolation processing, for example, to form the cavity line 52. Alternatively, the cavity line 52 may be formed along the boundary between the heart cavity and the cardiac muscle which is specified by image processing such as binarization processing with respect to the image data 22.

The inflow line 54 and the outflow line 56 are set by a user, such as a doctor. The user, such as a doctor, designates locations of start points S and end points E on the display image corresponding to the image data 22, thereby setting the inflow line 54 and the outflow line 56, for example.

After initial setting of the inflow line 54 and the outflow line 56 by the user, the region of interest setting unit 50 modifies the inflow line 54 to connect with the cavity line 52 and modifies the outflow line 56 to connect with the cavity line 52, and further connects the inflow line 54 and the outflow 56.

The region of interest setting unit 50, for example, shifts the start point S of the inflow line 54 to a location of the sample point on the cavity line 52 which is the closest to the start point S. The region of interest setting unit 50 further shifts the end point E of the outflow line 56 to a location of the sample point on the cavity line 52 which is the closest to the end point E. The region of interest setting unit 50 further forms a straight line or a curved line connecting the end point E of the inflow line 54 with the start point S of the outflow line 56.

The region of interest setting unit 50 thus forms a region enclosed by the cavity line 52, the inflow line 54, and the outflow line 56, and sets that region as a region of interest.

Once the region of interest corresponding to the heart cavity has been set, the energy computation unit 70, based on the velocity information of blood flow in the region of interest corresponding to the heart cavity, calculates an amount of energy lost in the blood flow in the heart cavity.

The energy computation unit 70, based on the following expressions, calculates an energy loss for each of a plurality of sample points within the region of interest, and further calculates a sum total of the energy loss at the plurality of sample points within the region of interest.

$$[ Mathematical Expression 1 ]$$

$${μ ( \frac{δ {u}_{i}}{δ {x}_{j}} + \frac{δ {u}_{j}}{δ {x}_{i}} )}^{2} … energy loss at each sample point$$

$$\int {μ ( \frac{δ {u}_{i}}{δ {x}_{j}} + \frac{δ {u}_{j}}{δ {x}_{i}} )}^{2} d v … sum total of energy loss within a region of interest$$

$$μ \text{:} coefficient of viscosity of blood$$

$$δ ui \text{:} change in flow rate in i \text{-} direction$$

$$δ uj \text{:} change in flow rate in j \text{-} direction$$

$$δ xi \text{:} change in distance in i \text{-} direction$$

$$δ xj \text{:} change in distance in j \text{-} direction$$

In calculation using the xy coordinate system, the i-direction corresponds to the x-direction and the j-direction corresponds to the y-direction, for example, in Mathematical Expression 1. In calculation using the rθ coordinate system, the i-direction corresponds to the r-direction and the j-direction corresponds to the θ-direction, for example. In calculation using the rθ coordinate system, Mathematical Expression 1 may be modified to a mathematical expression suitable for the rθ coordinate system.

The energy computation unit 70, based on Mathematical Expression 1, for example, calculates a sum total of the energy loss for each time phase over a plurality of time phases.

The energy computation unit 70 further calculates inflow energy which is kinetic energy of blood flow flowing into the heart cavity based on the velocity information of the blood flow corresponding to the inflow line 54, and further calculates outflow energy which is kinetic energy of blood flow flowing out of the heart cavity based on the velocity information of the blood flow corresponding to the outflow line 56.

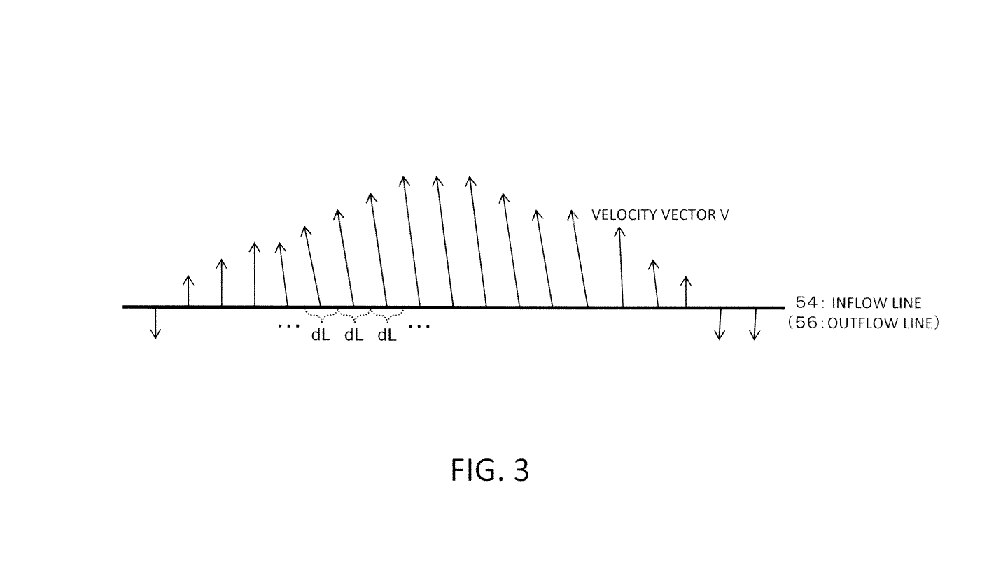

FIG. 3 is a diagram for explaining an example for calculation of the inflow energy and the outflow energy. To calculate the inflow energy, the energy computation unit 70 first divides the inflow line 54 into a plurality of sample straight lines (lengths dL). The length dL of each sample straight line is preferably about one pixel of the image data, for example.

The energy computation unit 70 then obtains, for each sample straight line, a velocity vector V on the sample straight line. For example, the velocity vector V on a single sample point on each sample straight line, such as a center point of the sample straight line, is designated as the velocity vector V of the sample straight line. Of course, an average of velocity vectors V at a plurality of sample points on each sample straight line may be used as the velocity vector V of the sample straight line.

The energy computation unit 70 further calculates the kinetic energy for each sample straight line, based on the velocity vector V of the sample straight line, according to the following expression.

½mV² . . . kinetic energy of each sample straight line [Mathematical Expression 2]

m=p×dL×V

V: magnitude of velocity vector

ρ: blood density

d L: length of sample straight line

ρ=1060 Kg/m³

The energy computation unit 70 then calculates a sum total of kinetic energy for a plurality of sample straight lines forming the inflow line 54, as the inflow energy. The energy computation unit 70 thus calculates the inflow energy for each time phase over a plurality of time phases.

Further, to calculate the outflow energy, the energy computation unit 70 divides the outflow line 56 into a plurality of sample straight lines (lengths dL), and calculates the outflow energy according to processing similar to that for calculating the inflow energy. Specifically, the energy computation unit 70 calculates kinetic energy for each sample straight line of the outflow line 56 according to Mathematical Expression 2, calculates a sum total of the kinetic energy for the plurality of sample straight lines forming the outflow line 56, and designates the sum total as the outflow energy. The energy computation unit 70 calculates the outflow energy for each time phase over a plurality of time phases.

Here, only the inflow line 54 may be set for calculating only the inflow energy, or only the outflow line 56 may be set for calculating only the outflow energy.

Once the energy computation unit 70 obtains calculated values of the energy loss, the inflow energy, and the outflow energy, the display processing unit 80 forms a display image indicating these calculated values. The display processing unit 80 forms, for example, a graph showing a change in the energy loss with time, as the display image.

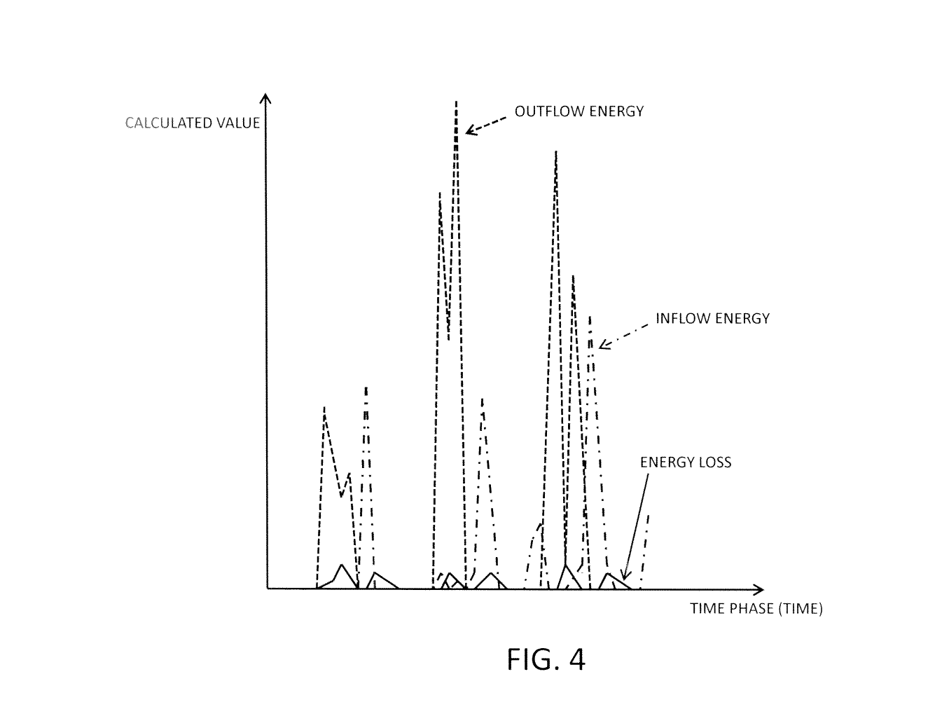

FIG. 4 illustrates a specific example graph showing a change in the energy loss with time. The graph illustrated in FIG. 4 shows the calculated values on the vertical axis with respect to a time axis corresponding to the horizontal axis.

The display processing unit 80 forms a graph showing a calculated value of the energy loss calculated for each time phase (a sum total within the region of interest) over a plurality of time phases. Consequently, the waveform of the energy loss indicated by a solid line in FIG. 4 is obtained.

The display processing unit 80 further forms a graph showing a calculated value of the inflow energy calculated for each time phase (a sum total on the inflow line) over a plurality of time phases and a graph showing a calculated value of the outflow energy calculated for each time phase (a sum total on the outflow line) over a plurality of time phases. Consequently, the waveform of the inflow energy indicated by a dashed and single-dotted line in FIG. 4 and the waveform of the outflow energy indicated by a dashed line in FIG. 4 are obtained.

The waveforms of the inflow energy, the outflow energy, and the energy loss are preferably indicated on the same time axis: that is, the respective calculated values corresponding to the same time phase are preferably correlated to the same time phase. Of course, the waveforms of the inflow energy, the outflow energy, and the energy loss may also be displayed individually.

The display processing unit 80 may further apply to the image data of an ultrasound image concerning the heart cavity, for each sample point within the region of interest, display processing in accordance with the energy loss for the sample point, to form a display image showing the distribution state of the energy loss within the heart cavity.

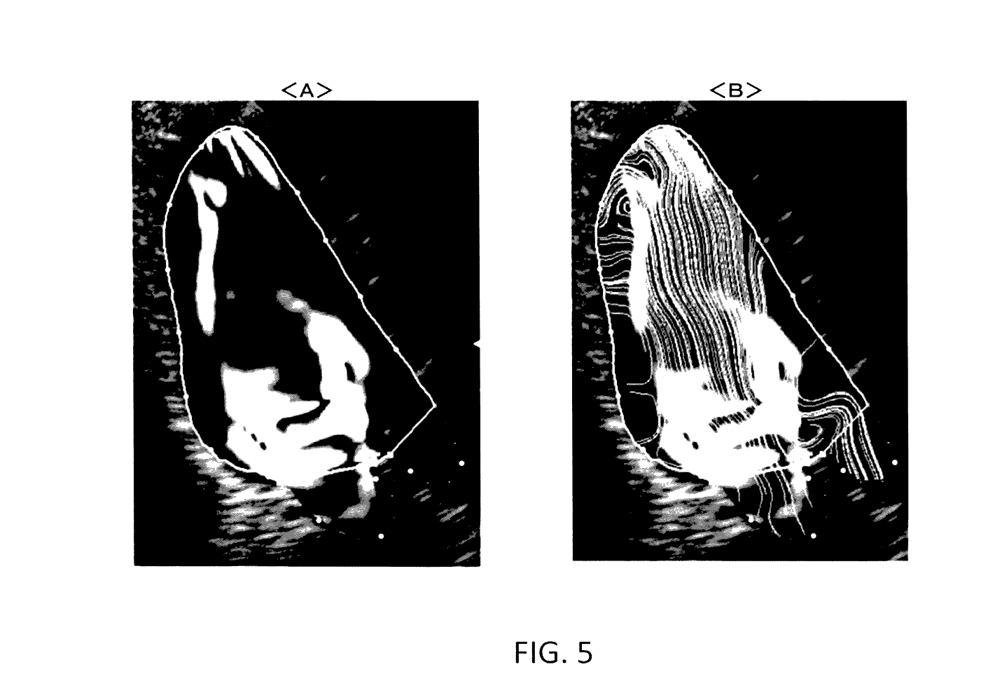

FIG. 5 illustrates a specific example display image regarding a distribution of energy loss. FIG. 5A illustrates a display image obtained by applying, to the ultrasound image (B-mode image) concerning the heart cavity to which a region of interest is set, coloring processing based on a hue in accordance with the energy loss value at each of a plurality of sample points within the region of interest.

The display processing unit 80 applies coloring processing using yellow as a base color to an image portion at each sample with a relatively large energy loss value and applies coloring processing using red as a base color to an image portion at each sample with a relatively small energy loss value energy, for example, thereby forming the display image illustrated in FIG. 5A. Of course, colors other than yellow and red may be used as base colors to represent the energy loss values, or the degree of luminance or a change of patterns, for example, may be used to represent the energy loss values.

The display processing unit 80 may further form streamlines showing blood flow based on the velocity vectors at a plurality of sample points within the region of interest, and display the streamlines superposed on the image illustrated in FIG. 5A, thereby forming an image illustrated in FIG. 5B.

The streamline can be obtained by tracking blood flow in accordance with a distribution of two-dimensional velocity vectors from each of a plurality of start points, for example. For example, tracking starts from each start point in the direction of the velocity vector of blood flow at the location of the start point to search a tracking point, and tracking is further continued in the direction of the velocity vector of the blood flow at the tracking point to search the next tracking point. The tracking points TP are thus sequentially searched in accordance with the distribution of velocity vectors of blood flow for each start point, and a streamline of the blood flow is formed. In this manner, a plurality of streamlines corresponding to a plurality of start points can be formed as illustrated in FIG. 5B.

The kinetic energy to be exerted on the heart cavity will be described hereinafter. The energy computation unit 70 calculates the kinetic energy to be exerted on the heart cavity based on the velocity information on the cavity line.

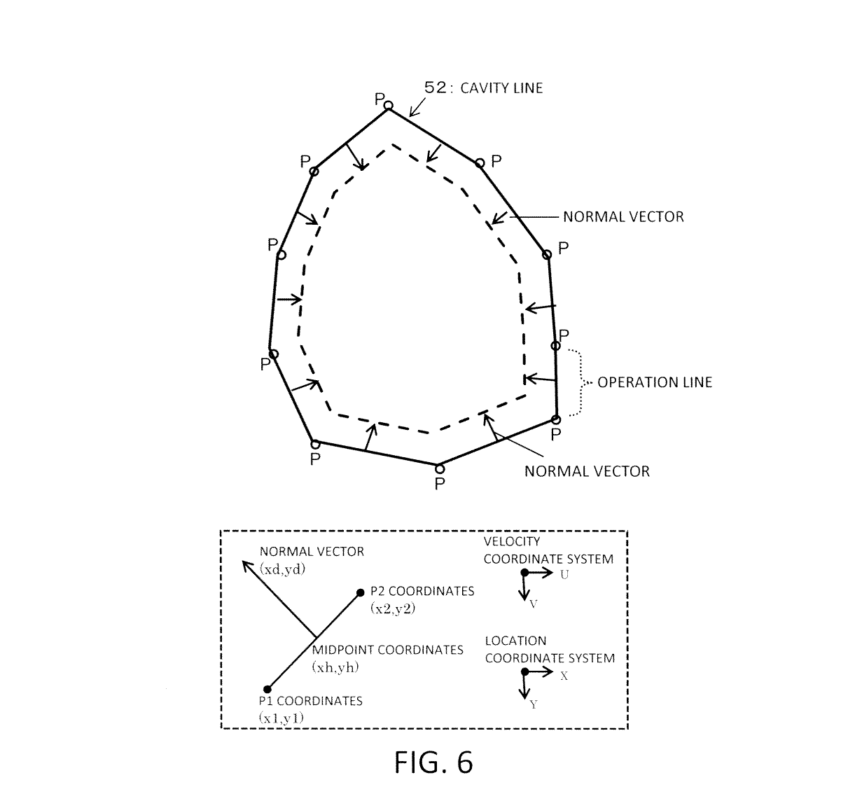

FIG. 6 is a diagram illustrating a specific example for calculating the kinetic energy from the cavity line 52. A plurality of trace points P on the cavity line 52 are used for calculating the kinetic energy. A straight line connecting two adjacent trace points (P1 and P2) is designated as an operation line, and a normal vector (xd, yd) orthogonal to the operation line is set from a midpoint (xh, yh), which is an initial point, of the operation line. For each operation line, the kinetic energy is calculated according to the following expression.

$$[ Mathematical Expression 3 ]$$

$$\frac{\frac{1}{2} ρ {\sum }^{PxlNum} ( {U}^{2} + {V}^{2} )}{total of samples} Q … total kinetic energy on each operation line$$

$$ρ = 1060 Kg \text{/} {m}^{3}$$

$$ρ \text{:} blood density$$

$$U \text{:} U \text{-} direction component of velocity vector at each sample point$$

$$V \text{:} V \text{-} direction component of velocity vector at each sample point$$

$$variation of area Q = ( xd \times wU ) + ( yd \times wV )$$

$$wU \text{:} U \text{-} direction component of velocity vector at midpoint$$

$$wV \text{:} V \text{-} direction component of velocity vector at midpoint \\ ( unit of kinetic energy \text{:} density * square of velocity * velocity * distance = \frac{Kg}{{m}^{3}} \frac{{m}^{2}}{{s}^{2}} \frac{m}{s} m = \frac{Kgm}{{s}^{3}} = \frac{{Kgm}^{2}}{{s}^{3}} \frac{ }{m} = \frac{W}{m} )$$

According to Mathematical Expression 3, the kinetic energy is calculated for each of a plurality of sample points on the operation line based on the velocity vectors (U, V), and the sum total of the kinetic energy concerning the plurality of sample points on the operation line is divided by a total number of samples, which is further multiplied by an area change Q. The area change Q can be obtained by the inner product ((xd×wU)+(yd×wV)) of the velocity vectors (wU, wV) and the normal vectors (xd, yd) at the midpoint on the operation line.

The velocity vectors (U, V) at each sample point on the operation line may be calculated by pattern matching using image data on the operation line (on the cavity line 52) or calculated based on the velocity vectors of nearby blood flow on the operation line.

The energy computation unit 70 calculates the kinetic energy based on Mathematical Expression 3 for each of a plurality of operation lines within the cavity line 52. The energy computation unit 70 further calculates a sum total of the kinetic energy obtained from, for example, the plurality of operation lines (preferably, all the operation lines), and designates the sum total as the total kinetic energy to be exerted on the heart cavity. The energy computation unit 70 calculates the total kinetic energy for each time phase over a plurality of time phases.

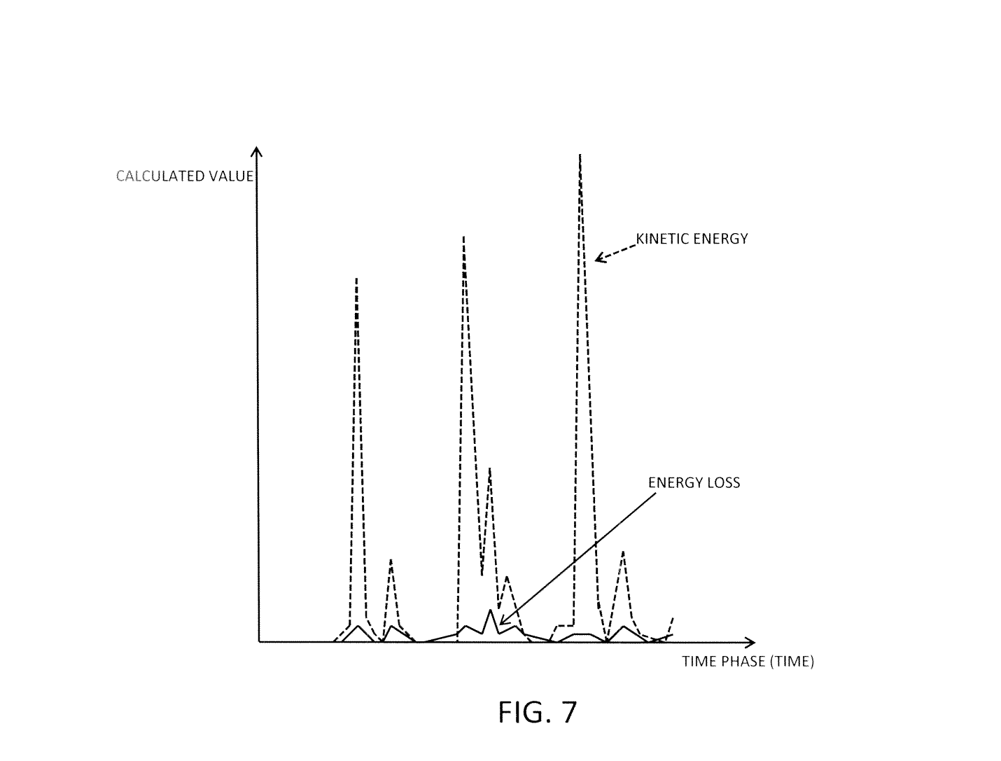

FIG. 7 illustrates a specific example graph showing a change in the total kinetic energy with time. The graph illustrated in FIG. 7 shows calculated values on the vertical axis with respect to a time axis corresponding to the horizontal axis.

The display processing unit 80 forms a graph showing, over a plurality of time phases, the total kinetic energy calculated for each time phase, thereby obtaining the waveform of the kinetic energy indicated by a dashed line in FIG. 7. The display processing unit 80 may form a graph showing, over a plurality of time phases, the calculated value of the energy loss obtained for each time phase and indicate the waveform of the energy loss (in a solid line) along with the waveform of the kinetic energy (in a dashed line), as illustrated in FIG. 7, or may, of course, indicate only the waveform of the kinetic energy individually. Alternatively, the display processing unit 80 may indicate a ratio of the energy loss to the kinetic energy (energy loss/kinetic energy) which is calculated as an index value for evaluating the state of burden of the heart.

Referring back to FIG. 6, to calculate the total kinetic energy, the energy computation unit 70 may calculate total kinetic energy based on the velocity information toward the inside of the heart cavity and total kinetic energy based on the velocity information toward the outside of the heart cavity.

For example, in Mathematical Expression 3, the sum total of the kinetic energy concerning a plurality of operation lines, which causes a positive (plus) area change Q and the sum total of the kinetic energy concerning a plurality of operation lines, which causes a negative (minus) area change Q, may be individually calculated. Further, to form the waveform of the kinetic energy, of the sum total of kinetic energy causing a positive area change Q and the sum total of kinetic energy causing a negative area change Q, the sum total having the greater absolute value may be selected for each time phase, to thereby form the kinetic energy waveform over a plurality of time phases. Of course, the sum of the sum total of kinetic energy with a positive area change Q and the sum total of kinetic energy with a negative area change Q or a difference between the two sum totals may be calculated for each time phase, to thereby form the waveform of the sum value or difference value.

The ultrasonic diagnostic device illustrated in FIG. 1 can be used to, for example, determine, based on the inflow energy and the outflow energy, whether or not the flow rate of blood flow is sufficient, and further determine, based on the energy loss, whether or not the burden of the heart (cardiac burden) is large.

For example, it is anticipated, from the inflow energy and the outflow energy which are relatively great, and greater than a reference value, that a sufficient flow rate of blood flow can be maintained within the body of the examinee. Further, relatively small energy loss within the blood flow of the heart cavity, obtained along with a sufficient flow rate of blood flow, can indicate a high possibility of the heart being normal. Even a sufficient flow rate of blood flow, however, when maintained with a large energy loss within the blood flow of the heart cavity, which is larger than a reference value, may force the heart to supply large kinetic energy to the blood flow to maintain such a sufficient flow rate of the blood flow, imposing a significant burden on the heart (cardiac burden).

The example diagnosis described above is only one example. Further, diagnosis using the ultrasonic diagnostic device illustrated in FIG. 1 should be performed carefully under the guidance of an expert such as a doctor.

Embodiments of the ultrasonic diagnostic device suitable for implementation of the present invention have been described above. At least one of the velocity vector computation unit 40, the region of interest setting unit 50, the cavity line velocity computation unit 60, the energy computation unit 70, and the display processing unit 80, for example, illustrated in FIG. 1 may be implemented by a computer, and the computer may be caused to function as an information processing device according to the present invention.

It should be noted that the embodiments described above are only examples in all respects and will not limit the scope of the present invention. The present invention may include various modifications within a scope which does not depart from its sprit.

### REFERENCE SIGNS LIST

10 probe, 12 transmitter and receiver unit, 20 image forming processing unit, 30 Doppler processing unit, 40 velocity vector computation unit, 50 region of interest setting unit, 60 cavity line velocity computation unit, 70 energy computation unit, 80 display processing unit, 82 display unit, 90 control unit.

1. An ultrasonic diagnostic device comprising:
a probe configured to transmit and receive an ultrasonic wave;
a transmitter and receiver unit configured to control the probe to obtain a received signal of an ultrasonic wave from an interior of an organism;
a Doppler processing unit configured to obtain Doppler information of the interior of the organism based on the received signal of an ultrasonic wave;
a velocity information generating unit configured to obtain velocity information of blood flow based on the Doppler information of the interior of the organism;
an image forming processing unit configured to obtain image data concerning the interior of the organism based on the received signal of an ultrasonic wave;
a region of interest setting unit configured to set, in the image data, a region of interest corresponding to a heart cavity;
an energy computation unit configured to calculate an amount of energy lost within blood flow in the heart cavity based on the velocity information of blood flow within the region of interest corresponding to the heart cavity; and
a display processing unit configured to form a display image showing the amount of lost energy which is calculated.

2. The ultrasonic diagnostic device according to claim 1, wherein
the energy computation unit is configured to calculate the amount of lost energy for each of a plurality of sample points within the region of interest corresponding to the heart cavity.

3. The ultrasonic diagnostic device according to claim 2, wherein
the display processing unit is configured to apply, to the image data concerning the interior of the organism at each of the sample points within the region of interest, display processing in accordance with the amount of lost energy of the sample point, to form a display image showing a state of distribution of the amount of lost energy within the heart cavity.

4. The ultrasonic diagnostic device according to claim 2, wherein
the energy computation unit is configured to calculate a sum total of the amount of lost energy within the region of interest, based on the amount of lost energy at the plurality of sample points within the region of interest corresponding to the heart cavity.

5. The ultrasonic diagnostic device according to claim 4, wherein
the display processing unit is configured to form a graph indicating, over a plurality of time phases, the amount of lost energy which is calculated for each time phase.

6. The ultrasonic diagnostic device according to claim 1, wherein
the region of interest setting unit is configured to designate a region, in the image data, enclosed by a cavity line which is set as an outer edge of the heart cavity, as the region of interest.

7. The ultrasonic diagnostic device according to claim 6, wherein
the region of interest setting unit is configured to designate a region, in the image data, enclosed by an inflow line which is set in a flow path of blood flow flowing in the heart cavity, an outflow line which is set in a flow path of blood flow flowing out of the heart cavity, and the cavity line which is set as an outer edge of the heart cavity, as the region of interest.

8. The ultrasonic diagnostic device according to claim 1, wherein
the energy computation unit is configured to calculate inflow energy that is kinetic energy of blood flow flowing into the heart cavity, based on the velocity information of blood flow corresponding to an inflow line which is set in a flow path of blood flow flowing into the heart cavity and calculate outflow energy that is kinetic energy of blood flow flowing out of the heart cavity, based on the velocity information of blood flow corresponding to an outflow line which is set in a flow path of blood flow flowing out of the heart cavity, and
the display processing unit is configured to form a graph showing at least one of the inflow energy and the outflow energy calculated for each time phase, over a plurality of time phases.

9. The ultrasonic diagnostic device according to claim 1, wherein,
the region of interest setting unit is configured to designate, as the region of interest, a region, in the image data, enclosed by an inflow line which is set in a flow path of blood flow flowing into the heart cavity, an outflow line which is set in a flow path of blood flow flowing out of the heart cavity, and the cavity line which is set as an outer edge of the heart cavity, and
the energy computation unit is configured to calculate inflow energy that is kinetic energy of blood flow flowing into the heart cavity, based on the velocity information of blood flow corresponding to the inflow line and calculate outflow energy that is kinetic energy of blood flow flowing out of the heart cavity, based on the velocity information of blood flow corresponding to the outflow line.

10. The ultrasonic diagnostic device according to claim 1, wherein
the velocity information generating unit is configured to generate velocity information of at least one of blood flow and cardiac muscle on a cavity line which is set as an outer edge of the heart cavity, based on at least one of the Doppler information and the image data concerning the interior of the organism, and
the energy computation unit is configured to calculate kinetic energy to be exerted on the heart cavity, based on the velocity information on the cavity line.

11. The ultrasonic diagnostic device according to claim 10, wherein
the velocity information generating unit is configured to generate the velocity information of blood flow or cardiac muscle for each of a plurality of sample points on the cavity line, and
the energy computation unit is configured to calculate a sum total of the kinetic energy to be exerted on the heart cavity based on the velocity information at the plurality of sample points on the cavity line.

12. The ultrasonic diagnostic device according to claim 11, wherein
the energy computation unit is configured to calculate, from the velocity information at the plurality of sample points on the cavity line, a sum total of the kinetic energy based on the velocity information toward inside of the heart cavity and a sum total of the kinetic energy based on the velocity information toward outside of the heart cavity.

13. The ultrasonic diagnostic device according to claim 1, wherein
the region of interest setting unit is configured to designate a region in the image data enclosed by a cavity line which is set as an outer edge of the heart cavity, as the region of interest,
the velocity information generating unit is configured to generate velocity information concerning at least one of blood flow and cardiac muscle on the cavity line based on at least one of the Doppler information and the image data concerning the interior of the organism, and
the energy computation unit is configured to calculate kinetic energy to be exerted on the heart cavity based on the velocity information on the cavity line.

14. The ultrasonic diagnostic device according to claim 13, wherein
the energy computation unit is configured to calculate a sum total of kinetic energy based on the velocity information toward inside of the heart cavity and a sum total of kinetic energy based on the velocity information toward outside of the heart cavity, from the velocity information at the plurality of sample points on the cavity line.

15. An information processing device, comprising:
a velocity information generating unit configured to obtain velocity information of blood flow based on Doppler information of an interior of an organism obtained using an ultrasonic wave;
a region of interest setting unit configured to set a region of interest corresponding to a heart cavity in image data concerning the organism obtained using an ultrasonic wave; and
an energy computation unit configured to calculate an amount of energy lost within blood flow in the heart cavity based on the velocity information of blood flow within the region of interest corresponding to the heart cavity.
